(19) United States
(12) Patent Application Publication
(10) Pub. No.: US 2009/0035216 A1
(43) Pub. Date: Feb. 5, 2009

(54) Method for determining in vivo biopharmaceutical concentration or bioavailability

(75) Inventors: Morten Svenson, Copenhagen (DK); Klaus Bendtzen, Lynge (DK)

Correspondence Address: EDWARDS ANGELL PALMER & DODGE LLP, P.O. BOX 55874, BOSTON, MA, 02205, US

(73) Assignee: BioMonitor Aps

(21) Appl. No.: 11/890,282

(22) Filed: Aug. 3, 2007

(51) Int. Cl. A61K 49/00; G01N 33/53; A61P 43/00; A61K 39/395

(52) U.S. Cl. 424/9.1; 435/7.8; 424/135.1

(57) ABSTRACT

The invention provides a method for determining the bioavailability of single light chain bio-agents in biological samples. The method determines that bioavailability by utilising a labelled form of the bio-agent and incubating with a probe that binds for the alternative light chain antibody form as compared to the form of the bio-agent, followed by a fractionation step where the probe, with any bound bio-agent, is isolated and the level of label from the labelled bio-agent is determined.

### FIELD OF INVENTION

The present inventions relates to the monitoring of the availability of monoclonal antibody bio-agents in vivo in patients who may have developed an antibody response to the bio-agent.

### BACKGROUND TO THE INVENTION

According to the Pharmaceutical Research and Manufacturers of America (PhRMA) millions of people have benefited from medicines and vaccines developed through biotechnology, and according to recent reports there are numerous further biopharmaceuticals for the treatment of more than 100 diseases currently in development. In their survey, the PhRMA identifies 324 biotechnology medicines in development for nearly 150 diseases. These include 154 medicines for cancer, 43 for infectious diseases, 26 for autoimmune diseases and 17 for AIDS/HIV and related conditions. These potential medicines, all of which are either in human clinical trials or under review by the Food and Drug Administration, will bolster the list of 108 biotechnology medicines already approved and available to patients. The report is available from http://www.phrma.org/new medicines in development for biotechnology/, and is hereby incorporated by reference.

The widespread use of biopharmaceuticals raises the possibility that some patients may develop antibodies to the drugs, which can greatly decrease the efficacy of the (biopharmaceutical) drug, or completely obliterate the benefit of taking the drug, resulting in considerable wasted expenditure on ineffective therapy, and more importantly, lost time in the treatment of the disorder which can have catastrophic effects in terms of the development of disease and disorders in the patient. Indeed, response failure due to induction of antibodies (Abs) against biopharmaceuticals is increasingly being realized. The development of host antibodies can be remedied by increasing dosage—although this is typically a delayed and rather temporary response as the prescription dosage is typically only increased once patient symptoms noticeably deteriorate, and the increased dosage may well result in further augmentation of the patients immune system. There is therefore a need for methods to determine the bioavailability of biopharmaceutical drugs.

The development of host (patient) antibodies against biopharmaceutical use is particularly of issue when the drug is delivered chronically, i.e. periodic administration over a period of months or years.

Anti-TNF alpha drugs are among important group of this type of biopharmaceuticals.

Anti-tumor necrosis factor (TNF) therapy has become an important alternative in the management of several chronic immunoinflammatory diseases. Three recombinant anti-TNF drugs are currently approved for clinical use in patients with various chronic inflammatory diseases such as rheumatoid arthritis, Crohn's diseases and severe psoriasis: 1) Remicade™ (infliximab), a mouse-human IgG1-kappa anti-TNF-alpha monoclonal antibody, 2) Enbrel™ (etanercept), a fusion protein of human TNF receptor 2 and human IgG1, and 3) Humira™ (adalimumab), a fully human IgG1-kappa anti-TNF-alpha monoclonal antibody. Two other anti-TNF-alpha antibody constructs have shown promise in pivotal phase III trials in patients with some of the same diseases: 4) Cimzia™ CDP870 (certolizumab pegol), a PEGylated Fab fragment of a humanized anti-TNF-alpha monoclonal antibody, and 5) CNTO 148 (golimumab), a fully human IgG1-kappa anti-TNF-alpha monoclonal antibody. All of these proteins dramatically lower disease activity and, in some patients, may induce remission. Unfortunately, however, not all patients respond favorably to anti-TNF antibodies. Some patients either do not respond at all (primary response failure) or they respond initially but have later relapses (secondary response failure) despite increased dosage and/or more frequent administration of the drugs. The reason(s) for these response failures are not always clear but interindividual and even intraindividual differences in bioavailability and pharmacokinetics may contribute to the problem. Immunogenicity of the drugs causing patients to develop anti-antibodies is a problem now recognized by many investigators, drug-controlling agencies, health insurance companies and drug manufacturers. Monitoring of patients for circulating levels of functional anti-TNF drugs and anti-antibody development is therefore warranted so that administration can be tailored to the individual patient and so that prolonged therapies can be provided effectively and economically with little or no risk to the patients.

By way of a non limiting example, we refer to Infliximab (Remicade®).

Infliximab is a mouse-human chimeric monoclonal IgG antibody against tumor necrosis factor-alpha (TNF-alpha), in combination with methotrexate, is approved for the treatment of moderate-to-severe rheumatoid arthritis (RA) in patients who have an inadequate response to one or more disease-modifying antirheumatic drugs (DMARD). In randomized clinical trials, intravenous infusions of infliximab, 3 mg/kg every 4 to 8 weeks, induce a positive response at 30 weeks in approximately 55% of patients and the response can be maintained in many patients with repeated infusions [1-4].

With repeated infusions, however, the formation of neutralizing anti-infliximab antibodies becomes a problem requiring increased doses or more frequent drug administration and may necessitate discontinuation of therapy because of secondary response failure and/or infusion-related side effects; this has been observed in both RA patients and in patients with other immunoinflammatory diseases [5-13]. Our own clinical experience, for example, rapidly showed that the generally recommended dosage of 3 mg/kg at weeks 0, 2, 6 and every 8 weeks thereafter was inadequate in a large proportion of patients. The mean weekly dosages per patient of infliximab at 3 year follow up were 35 (n=5), 54 (n=35), 44 (n=26), and 38 (n=17) mg at years 2002, 2003, 2004, and 2005, respectively. Furthermore, the recommended dose regimen was originally established on the basis of clinical trials using relatively large cohorts of RA patients of both sexes, with differences in age, co-morbidities and concurrent therapies [4]. In clinical practice, however, patients with RA or any other chronic inflammatory disease treated with infliximab may differ considerably from the average patient in randomized clinical trials [14]. For example, even though the initial bioavailability of infliximab approaches 100% because of the intravenous administration of the drug, differences in pharmacokinetics may result in individual patients having inadequate drug levels for extended periods of time between infusions. This problem can be exaggerated by the appearance of antibodies. Indeed, response failures are frequent, and development of assays that can be used to monitor bioavailability and Ab development is of direct clinical importance.

There is therefore a problem with the use of biopharmaceuticals that the patient's immune system can develop an antibody response, and this problem can result in ineffective patient treatment and increased costs of treatment.

A number of studies have reported a concentration-effect relationship of therapeutic proteins directed against TNF-alpha in patients with RA and Crohn's disease [12-16]. Thus, high serum concentrations of anti-TNF antibody constructs just before an infusion, i.e. high trough levels, are associated with clinical improvement, whereas low through levels are associated with poor clinical response.

Several different methods have been used to assess circulating levels of anti-TNF biopharmaceuticals. Most of these are based on enzyme immunoassays (EIA) where the IgG construct, bound to TNF-alpha immobilized on plastic beads or wells, is detected with rabbit or goat anti-human IgG-Fc antibody [14, 17].

Certain embodiments of the present invention are disclosed, by the same inventors as the present invention, in Bendtzen et al., Arthritis & Rheumatism Vol 54, No 12, published December 2006, which is hereby incorporated by reference.

The present invention provides a highly effective and sensitive assay for detection of the in vivo bioavailability or concentration of a very important class of biopharmaceutical, monoclonal antibodies.

### SUMMARY OF THE INVENTION

The present invention provides a method for determining the concentration or bioavailability of a single light chain subtype bio-agent in a biological sample obtained from a subject, said method comprising
a. Obtaining the single light chain subtype bio-agent in a form which comprises a detectable label;
b. admixing the biological sample with the labelled bio-agent and a probe which selectively binds the alternative single light chain subtype to form an incubation mixture;
c. isolating a fraction which is enriched for the probe and/or probe bound immunoglobulins from the incubation mixture formed in step b.;
d. measuring a signal from the detectable label present in the isolated fraction to determine the concentration or bioavailability of the single light chain subtype bio-agent in the biological sample.

Step b. typically further comprises a period of incubation of the incubation mixture prior to the fractionation step c).
The invention further provides for a kit comprising:
   a. A single light chain subtype bio-agent in a form which comprises a detectable label,
   b. A probe which selectively binds the alternative single light chain subtype to the bio-agent.
The invention further provides for a method of treatment of a disease with a single light chain subtype biopharmaceutical, said method comprising performing the above method for determining the concentration or bioavailability of a single light chain subtype biopharmaceutical in a biological sample obtained from a subject, on a patient to determine whether the patient requires either an altered dosage regime of the biopharmaceutical or alternative pharmaceutical therapy.
The invention further provides for a method for determining the suitability or accuracy of an in vivo diagnostic method which involves the administration of a labelled bio-agent to a subject, said method comprising the above method for determining the concentration or bioavailability of a single light chain subtype bio-agent in a biological sample obtained from a subject, to determine whether the patient has (either innate or developed) an immune response to the bio-agent.
The invention further provides for a method for the measurement of host derived antibodies which recognise a single light chain subtype bio-agent in a biological sample obtained from a subject, said method comprising the following steps
   a. Obtaining the single light chain subtype bio-agent in a form which comprises a detectable label;
   b. admixing the biological sample with the labelled bio-agent and a probe which selectively binds the alternative single light chain subtype to form an incubation mixture;
   c. isolating a fraction which is enriched for the probe and/or probe bound immunoglobulins from the incubation mixture formed in step b.;
   d. measuring a signal from the detectable label present in the isolated fraction to measure the level of host derived antibodies which recognise a single light chain subtype bio-agent in the biological sample.

Step b. typically further comprises a period of incubation of the incubation mixture prior to the fractionation step c).

### BRIEF DESCRIPTION OF THE FIGURES

FIG. 1: Genetically engineered anti-TNF antibody constructs The upper panel shows the light- and heavy chain genes spliced together from TNF-alpha-immunized murine splenocytes (VL and VH segments) and from human IgG1 (CL, CH, Hinge, CH2, and CH3 segments). The chimeric protein, infliximab, is produced when the gene constructs are expressed in antibody-secreting immortalized myeloma cells.

### ABBREVIATIONS

VL and VH: Variable regions of IgG on light and heavy chains, respectively.

CL, CH, CH2 and CH3: Constant regions of IgG on light and heavy chains, respectively.

Fab: Fragment antigen binding, including the variable parts of IgG.

Fc: Human IgG1 Fc region.

CDR: Complementarity-determining regions.

FR: Framework regions.

mAb: Monoclonal antibody.

PEG: polyethylene glycol.

FIG. 2: Measurements of human antibodies against anti-TNF antibody constructs. A: Two examples of how to measure anti-antibodies by solid-phase ELISA, in this case antibodies directed against infliximab. The left part shows the detection of so-called human anti-chimeric antibodies (HACA), sometimes called human anti-mouse antibodies (HAMA), using wells coated with Fab fragments of the drug. As anti-antibodies in this setup may recognize human epitopes in the Fab fragments, being anti-human antibodies (HAHA), the terms HACA/HAMA may in some cases be misleading. The right part shows another ELISA for anti-antibodies against infliximab. Note that sera with rheumatoid factor are likely to cause false positive results in this assay. B: Fluid-phase RIA measuring anti-antibodies (all isotypes) from patients treated with infliximab. In this example, patient serum containing antibodies against infliximab is incubated overnight with purified ¹²⁵I-infliximab. Free and immunoglobulin-bound tracer are separated by affinity chromatography using matrix-bound anti-human lambda-chain antibody.

FIG. 3: Development of anti-infliximab antibodies and association with trough level TNF-alpha binding afforded by infliximab. Anti-infliximab antibody- and infliximab levels were measured in patient sera beginning immediately before the third drug infusion (after 1.5 months) and before infusions at time points 3 and 6 months, respectively. S-infliximab was quantified as the level of infliximab in a serum pool from healthy individuals providing the same TNF-alpha binding as the patient serum tested. Arrows: Responses in the patient testing positive before start of therapy.

FIG. 4. Dose-response curves of TNF-alpha binding capacities in sera added various amounts of infliximab. Sera from 5 patients treated for 6 months with infliximab were assessed (closed circles): 2 had high antibody activity (Ab++), 1 had low antibody activity (Ab+), and 2 had no detectable antibody activity. The sera were compared with a serum pool from 30 untreated healthy individuals (open circles). The amounts of added infliximab corresponded to the levels observed in patient sera (FIG. 3). Horizontal line shows 50% of maximum TNF-alpha binding afforded by added infliximab.

FIG. 5: Relations to clinical outcome. A: Trough TNF-alpha binding capacities due to infliximab (s-infliximab) in sera drawn immediately before the third drug infusion (after 1.5 months). The data are related to patients receiving standard dosage of the drug throughout the observation period (no failure) and to the numbers of patients with therapeutic failure within various time points after start of therapy. B: Antibody levels in sera drawn before the fourth infliximab infusion (after 3 months). The data are related to patients on continued standard dosage and to patients in need of increased dosage of infliximab within the observation period. C: Antibody levels as in part B. The data are related to patients on continued therapy (ongoing) and to patients where therapy for various reasons had to be stopped within the observation period. P values are shown (Mann-Whitney rank sum test).

FIG. 6: Initial factors influencing antibody development. A: The left column shows TNF-alpha binding capacities due to infliximab (s-infliximab) in sera drawn immediately before the third infliximab infusion (after 1.5 months). The data are related to, and paired with, the levels of antibodies after 6 months on infliximab (right column). A reverse linear correlation was observed: Spearman r=−0.40 (P=0.0007). B: Trough TNF-alpha binding capacities due to infliximab (s-infliximab) in sera drawn immediately before the third infliximab infusion (after 1.5 months). The data are related to patients who before start of treatment had low or high (upper quartile) plasma levels of C-reactive protein (CRP). C: Antibody levels in sera drawn before the fourth infliximab infusion (after 3 months). The data are related to patients who before start of treatment had low or high (upper quartile) disease activity scores (DAS28). P values are shown (Mann-Whitney rank sum test).

### DETAILED DESCRIPTION OF THE INVENTION

### Definitions

The term “bio-agent” refers to either a single light chain biopharmaceutical or a single light chain biodiagnostic. The bio-agent typically consists of an intact light chain immunoglobulin, or a fragment thereof which comprises at least a variable domain, and at least part of the light chain constant region. The bio-agent is typically free of heavy chain immunoglobulins. Table 1 provides a list of bio-agents which comprise of monoclonal antibodies, including those whose in vivo concentration/bioavailability may be determined using the methods of the present invention.

Heavy chain antibodies typically have a molecular weight of approximately 50 kDa, whereas the light chains typically have a molecule weight of approximately 25 kDa. The light and heavy chains are joined together by a disulfide bond near the carboxyl terminus of the light chain. The heavy chain is divided into an Fc portion, which is at the carboxyl terminal (the base of the Y), and a Fab portion, which is at the amino terminal (the arm of the Y). Carbohydrate chains are attached to the Fc portion of the molecule. The Fc portion of the Ig molecule is composed only of heavy chains. The Fc region contains protein sequences common to all Igs as well as determinants unique to the individual classes. These regions are referred to as the constant regions because they do not vary significantly among different Ig molecules within the same class. The Fab portion of the Ig molecule contains both heavy and light chains joined together by a single disulfide bond. One heavy and one light chain pair combine to form the antigen binding site of the antibody. Human light chain antibodies can be of either lambda or kappa isotypes.

The term “intact light chain” refers to a polypeptide which consists of both one or more variable regions and a constant regions (or part thereof) a light chain isotype polypeptide. The intact light chain is the product of the expression of a light chain encoding polynucleotide, taking into account post-translational modifications which may occur during production within the expression system.

The “probe” selectively binds the alternative single light chain subtype to the bio-agent. Typically, the probe is an alternative form of a light chain antibody as compared to the bio-agent, for example, when the bio-agent is a lambda sub-type, the probe is a kappa sub-type and vice-versa. The probe may therefore be an anti-bio-agent light chain antibody which is of a different light-chain subtype as compared to the bio-agent. The probe may comprise a molecular or physical label which allows for it to be easily separated during step c), and in a preferred embodiment, the probe is immobilised on a solid support, such as an affinity matrix or magnetic bead. The probe may, however, comprise or be conjugated to an affinity molecule, such as a specific epitope (e.g. his tag) which can be subsequently used to fractionate the probe/probe bound immunoglobulin in step c. Alternatively the biotin/streptavidin system may be used—e.g. biotinylated probes may be captured using avidin/streptavidin coated beads, or a chromatography affinity matrix.

In one embodiment, the probe (selective binder to the alternative light chain subtype to the bio-agent) may be a functional bivalent, and therefore able to form large complexes with the target (bio-agent)—which may result in precipitation—allowing for fractionation of the probe and probe/target complexes.

Therefore, when the probe is an antibody (Fab2 or intact), immunoprecipitation can be performed. When the probe forms a precipitating complex with the target, precipitation can be made of the bio-agent anti-bio-agent antibody complexes by addition of the probe. If the probe does not form precipitation complexes (as often seen when using only one mAb), precipitation can be made by adding specific and precipitating antibodies against the probe.

Separation of the precipitate and supernatant may be made for example, by centrifugation or filtration.

Reference: Antibodies, A Laboratory Manual. Editors: Ed Harlow and David Lane. Cold Spring Harbor Laboratory, 1988. Chapter 11: Immunoprecipitation, page 424-468.

Therefore in one embodiment the fractionation step is performed by immunoprecipitation (antibody precipitation).

The term “biological sample” or “sample” refers to a sample which is obtained or derived from a patent which comprises patient derived immunoglobulin and may therefore be referred to as an immunoglobulin sample. By way of example, the sample may be selected from the group consisting of blood, blood serum, lymph fluid, lymph node tissue, spleen tissue, bone marrow, or an immunoglobulin enriched fraction derived from one or more of these tissues. In a preferred embodiment the sample is, or comprises blood serum or is an immunoglobulin enriched fraction derived from blood serum or blood. In one embodiment the sample is, or is derived from, a bodily fluid. In one embodiment the sample is derived (obtained) from body tissue. In one embodiment, the sample is obtained form a subject who has not recently been exposed to the bio-agent as the existence of a high concentration of the unlabelled bio-agent in the sample may effect the accuracy of the result obtained. In one embodiment, the sample is obtained form the subject prior to the planned administration of the bio-agent.

The term “subject” refers to the individual from which the biological sample is taken. Typically the subject is a patient who is either: (i) being considered for treatment, or undergoing treatment, or previously received treatment, wherein the treatment involves the administration of a monoclonal antibody based biopharmaceutical (bio-agent), or (ii) is being considered for diagnosis, or undergoing diagnosis, or has previously undergone diagnosis for a disorder or a disease, wherein the diagnosis involves the administration of a labelled (typically radio-labelled) monoclonal antibody into the body of the subject, wherein the monoclonal antibody (bio-agent) is used to specifically detect and/or localise the presence of the disorder or disease or disease causing agent. The patient may be an animal, such as a mammal, preferably a human being.

When we refer to “the concentration or bioavailability” herein, it refers to the determination of the concentration of bio-agent in the subject (such as in the sample, or tissue corresponding to the sample). The bio-agent concentration can be determined as the proportion of bio-agent which is free from host-immunoglobulins (i.e. bioavailable), compared to the amount which is associated with host-immunoglobulin. By comparing to data of the known (or predicted) concentration of the total concentration of bio-agent, the concentration of bio-available bio-agent, and/or host-immunoglobulin bound bio-agent can be determined.

The method of the invention allows for the measurement of the formation of labelled bio-agent—host derived immunoglobulin complexes—this is a measurement of the bioavailability of the bio-agent in vivo in the subject, and is also a measurement of the level of host derived immunoglobulins which recognise the bio-agent present in the subject (or biological sample).

The method of the present invention may be used therefore to determine (or predict) the level of bio-available bio-agent in the subject and/or the level of host-immunoglobulin bound bio-agent in the subject. The method of the invention also refers to a method for determining the presence of an immune response to the bio-agent in the subject, such as a method for determining the development of an immune response to the bio-agent in the subject. Such methods may be qualitative (i.e. presence of absence), or quantitative.

The method of the present invention may be used to determine the qualitative or quantitative level of host derived immunoglobulins which recognise the bio-agent present in the biological sample.

When referring to determining the concentration of a single light chain bio-agent, we refer to determination of the concentration of the single light chain bio-agent which is free—i.e. is not in the form of a complex with immunoglobulins derived from the biological sample. In this respect determination of the concentration of the single light chain bio-agent is a measure of the bioavailability of the bio-agent when used in vivo.

The method according to the invention may, for example, be used for identifying primary non- or low-responders, e.g. for treatment. These may, for example, be patients that happen to have an innate or a pre-developed immunoglobulin response to the bio-agent. Where the bio-agent is a diagnostic antibody, the identification of primary non- or -low responders can ensure the selection of a suitable diagnostic agent for each individual patient.

The method according to the invention may, for example, be used for identifying patients with secondary response failure. Secondary response failures can be asymptomatic, i.e. the only symptoms are that the treatment has become less effective or even non-effective. In this instance the use of the method according to the invention can be used to identify the development of secondary response failure before the patient or medical practitioner has noticed that the treatment is less effective. A higher dosage of treatment may be applied to ensure the correct in vivo concentration is achieved, or an alternative treatments can be selected, or a combination thereof. When the bio-agent is a diagnostic, the development of secondary response failure can be particularly catastrophic. Radio-labelled monoclonal antibodies are routinely used in the monitoring of diseases such as cancers, and some infectious diseases, where it is important to determine the size and/or location of the disease/agent—for example in identifying the presence/location of any secondary metastases. When the development of response failure (either primary or secondary) occurs unnoticed, the patient may be given the ‘all clear’—i.e. a false negative result, this can lead to the cessation of treatment and the latter re-appearance of the disease, often in a far more developed and possibly untreatable condition.

A further category of response failure is the development of (e.g. secondary) response failure associated with adverse side effects. Although rare, the development of a host-immune response in a subject can be accompanied by deleterious or unpleasant side effects. These may be caused by the development of antibodies which recognise the human or humanised bio-agents, but may then fail to distinguish with other host immunoglobulins. The present invention can therefore be used to prevent the administration of bio-agents to subjects who have either an innate or have previously developed an immune response to the bio-agent, subjects who may, for example, be vulnerable to adverse side effects associated with response failure.

In a highly preferred embodiment, the single light chain subtype bio-agent, such as biopharmaceutical, is a monoclonal antibody which comprises the lambda or kappa single light chain sub-type, but not both lambda and kappa single light chain sub-types.

In one embodiment, the single light chain subtype bio-agent, such as biopharmaceutical, is either a humanised of a fully-human monoclonal antibody.

In one embodiment the bio-agent is a biopharmaceutical.

In one embodiment, the biopharmaceutical is an antibody which specifically binds TNF-alpha (such as an anti-TNF alpha monoclonal antibody therapeutic).

In one embodiment, the bio-agent is a bio-diagnostic.

In one embodiment, the (immunoglobulin) sample is a biological fluid such as a blood or serum sample, or is derived there from.

In one embodiment, the sample may be selected from the group consisting of blood, blood serum, lymph fluid, lymph node tissue, spleen tissue, bone marrow, or an immunoglobulin enriched fraction derived from one or more of these tissues.

In one embodiment, the probe which selectively binds the alternative single light chain subtype is an immunoglobulin, such as the alternative subtype to the sub-type of the bio-agent (as referred to herein in the context of the bio-agent, such as biopharmaceutical).

In one embodiment, the detectable label is selected from the group consisting of: a radio label, a fluorescent label, a luminescent label.

In one embodiment, the incubation step b) is performed in a fluid phase.

In one embodiment, steps b. and c. comprises the sequential steps of (i) incubating the immunoglobulin sample with the labelled bio-agent and (ii) contacting the incubated immunoglobulin sample with the labelled bio-agent, with the probe.

In one embodiment, the probe is attached to a solid support which allows for the isolation of the fraction which is enriched for the probe and/or probe bound immunoglobulins.

In one embodiment step b. comprises incubating the sample the labelled bio-agent and a probe; wherein step c. is performed subsequent to step b, and; wherein step c. comprises isolating the fraction which is enriched for the probe and/or probe bound immunoglobulins.

In one embodiment, the isolation of the fraction which is enriched for the probe and/or probe bound immunoglobulins is based on affinity purification.

In one embodiment, the solid support is an affinity matrix, an affinity column matrix or a magnetic bead.

In one embodiment, step c) comprises a chromatographic step which enriches the probe and/or probe bound immunoglobulins on a basis of affinity.

In one embodiment, step c) comprises a chromatographic step which enriches the probe and/or probe bound immunoglobulins on a basis of immune precipitation.

In one embodiment, step c) comprises a chromatographic step which enriches the probe bound immunoglobulins on a basis of molecular weight (size) of the probe bound immuoglobulin (complex).

In one embodiment, the determination of the serum concentration or bioavailability in step b) is performed by comparing against control samples with predetermined or known concentration of the biopharmaceutical.

In one embodiment, the method comprises a radio-immuno assay (RIA), which may. For example, be performed in the liquid phase.

The invention also provides for a kit comprising: a single light chain subtype bio-agent (i.e. the bio-agent as referred to herein in a form which comprises a detectable label, and a probe which selectively binds the alternative single light chain subtype to the bio-agent, such as biopharmaceutical (such as the probe as described herein).

The kit according to the invention is typically accompanied by instructions for use in the method according to the invention. The bio-agent or biopharmaceutical can be as according to those described herein.

Clearly one major application area for the method of the present invention is in the selection and management of treatment regimes which involve the administration of monoclonal biopharmaceuticals to patients. Therefore, the method for determining the concentration or biological availability of a single light chain sub-type biopharmaceutical, as described herein, can be incorporated into a method of treatment of a disease or a disorder. By monitoring of the immunological status of the subject using the methods of the invention during the course of therapeutic treatment, the selection and/or administration of the biopharmaceutical agent can be tailored to ensure maximum therapeutic benefit to the patient, whilst ensuring cost effective use of expensive biopharmaceutical agents.

The method according to the invention may be used to determine whether the patient requires either an altered dosage regime of the biopharmaceutical or alternative pharmaceutical therapy.

Suitably the method may involve a periodic assessment of the serum concentration or bioavailability of the bio-agent in the patient.

The invention also refers to methods of determining whether a subject has developed an immune response to the bio-agent.

The invention provides for a method of determining whether the lack of treatment response in a patient is due to the formation of patient derived immunoglobulins against a single light chain subtype biopharmaceutical (using the method steps referred to herein).

The invention provides for a method of selecting the appropriate drug treatment for a patient suffering from a disease which is treatable with a single light chain subtype biopharmaceutical (using the method steps referred to herein).

The invention provides for a prognostic method for the determination of the likelihood of whether a patient will develop secondary response failure to a single light chain subtype biopharmaceutical (using the method steps referred to herein).

Obtaining the Single Light Chain Subtype Bio-Agent Agent in a Form which Comprises a Detectable Label: Labelling of Bio-Agents:

Numerous methods of labelling immunoglobulins are known in the art and can be used for the purposes of the present invention.

In one embodiment, the bio-agent is labelled by incubating the bio-agent with a labelled antigen, wherein the antigen is the specific antigen recognised by the bio-agent when used inside the body.

A preferred label is a radio label, which allow the labelled bio-agent to be used in radio-immuno assays. For example, ¹²⁵I labelling of Infliximab is described in Svensen et al., J. Clin Invest, 92, 2533-2539.

It is envisaged that by use of highly sensitive mass detection techniques, such as MALTI-TOF analysis, possibly in conjunction with immunoglobulin purification techniques, that the presence of the bio-agent—host immunoglobulin complex could be detected without the use of an exogenous label. For example, in step c. by using size exclusion chromatography, the free bio-agent and the host-immunoglobulin/probe associated bio-agent can be separated. The host-immunoglobulin associated bio-agent/probe (complex) can then be denatured (or a marker peptide from the bio-agent can be isolated), and the amount of ‘free’ bio-agent (i.e. not associated with host-immunoglobulin) and complexed bio-agent can then be compared by suitable protein quantification methods, such as by use of an immuno assay, or by MALDI-TOF, for example. Therefore, in one embodiment, it is not required to obtain an exogenously labelled bio-agent as the physical (chemical/immunological features (for example) of the bio-agent itself may be used as a label.

However, for ease of use, it is considered that the use of an exogenously labelled bio-agent is preferred—suitably the exogenous label is a distinct chemical or physical entity, not present in the unlabelled bio-agent, e.g. which may be incorporated into the bio-agent (such as a radio-labelled amino-acid), or is conjugated or otherwise attached to the bio-agent once the bio-agent has been prepared (e.g. a fluorescent or luminescent label).

In one embodiment the labelled bioagent is an ¹²⁵I labelled anti-TNF-alpha bio-agent, such as those described herein.

Incubating the Immunoglobulin Sample with the Labelled Bio-Agent and a Probe which Selectively Binds the Alternative Single Light Chain Subtype:

Once the labelled bio-agent has been obtained, it is incubated with the sample, typically in a suitable media, such as an assay buffer. The labelled bio-agent may be purified prior to incubation, e.g. by size-exclusion or molecular size-chromatography.

The incubation can occur in a fluid phase, or on a solid phase.

It is considered that the probe may also be added to the fluid phase which allows for, in the case of subjects who has developed an antibody response to the bio-agent, the formation of bio-agent-host immunoglobulin complexes which incorporate both the labelled bio-agent, the host immunoglobulins which recognise the labelled bio-agent, and the probe which also recognises the bio-agent. Subsequent isolation of the probe and/or probe bound immunoglobulins allows for the signal from the labelled bio-agent to be detected.

When in a liquid phase, both the labelled bio-agent and sample, and optionally the probe, are typically present in the fluid phase, such as in an assay buffer.

In one preferred embodiment, the probe may be attached to a solid phase, such as an affinity matrix/column support or bead. This facilitates the fractionation step c, as the host immunoglobulin bound labelled bio-agent will bind to the probe, which can easily be separated or fractionated by e.g. affinity chromatography or use of a magnet (referring to affinity matrix/support and magnetic bead embodiments, respectfully). Alternatively attachment of the probe to a (dense) particle, such as a bead can allow fractionation by, e.g. centrifugation or filtration.

One interesting embodiment is where the probe is biotinylated, and step c) involves the capture of the biointylated probe/probe bound immunoglobulin by an avidin/strptavidin bead, which can subsequently be isolated/fractionated) e.g. by use of a magnet (and magnetic beads) or other types of beads, such as those referred to herein.

We have found that the use of a liquid phase assay, where both labelled bio-agent and samples are present in a liquid phase, is particularly effective in providing a highly sensitive and accurate determination of concentration and/or bioavailability of bio-agents, and/or host immunoglobulins which recognise the bio-agents. Liquid phase assays typically are performed in the presence of a blocking agent, such as milk proteins or BSA to prevent or reduce non-specific binding. As described herein the liquid phase assay may be in the form of a radio-immuno assay (RIA).

In one embodiment the assay is performed in a fluid phase, such as a fluid phase radio-immunoassay.

In one embodiment the assay is performed in a solid phase immunoassay, such as using ELISA.

Subsequently Isolating a Fraction which is Enriched for the Probe and/or Probe Bound Immunoglobulins:

As described herein, the separation of the probe and/or probe bound immunoglobulins may be performed using numerous methods known in the art, typically based on molecular affinity:

As referred to above, in one embodiment, steps b. and c. comprise the sequential steps of (i) incubating the immunoglobulin sample with the labelled bio-agent and (ii) contacting the incubated immunoglobulin sample with the labelled bio-agent, with the probe. In such an embodiment, the probe is typically attached to a solid support which allows for the isolation of the fraction which is enriched for the probe and/or probe bound immunoglobulins. For instance, the probe may be attached to an affinity matrix as part of an affinity chromatography step—e.g. using an affinity column. Alternatively, the probe may be attached to a bead, such as a magnetic bead, for example.

In one embodiment step b. comprises incubating the sample, the labelled bio-agent and the probe; wherein step c. is performed subsequent to step b, and; wherein step c. comprises isolating the fraction which is enriched for the probe and/or probe bound immunoglobulins. For instance, the probe may be attached to a magnetic bead, where step c) involved fractionating the magnetic beads from the incubation matrix of step b), thereby isolating both the probe and probe/probe bound immunoglobulins. Alternatively the probe may comprise an affinity marker, such as a his-tag or a biotin marker (biotinylated probe), which can subsequently be used to isolate the probe and probe bound immunoglobulins.

As well as (dense) beads and magnetic beads, fluorescent, luminescent or coloured beads may also be used—these can be sorted using, for example, FACS.

Measuring a Signal from the Detectable Label Present in the Isolated Fraction to Determine the Serum Concentration or Bioavailability of the Single Light Chain Subtype Bio-Agent.

As the isolation of the fraction is step c) is performed by virtue of the probe, and the probe does not bind to the (labelled) bio-agent, the signal detected from the fraction (or fractions) obtained in step c) can only be derived from complexes where host immunoglobulin has recognised/bound to the bio-agent, and the probe has recognised/bound to the alternative single light chain sub-type present in the host-immunoglobulin.

The detection of the signal is therefore a measure of the level of host immunoglobulins present in the sample which recognise/bind to the bio-agent, and therefore is negatively correlated to the concentration of bio available bio-agent present in the sample.

Typically, step d) comprises a comparison step where data from one or more control sample(s) are used, which allows calibration of the data of the signal referred to in step d) to the data obtained from samples where the concentration of bio-agent is known, and/or the concentration of host-immunoglobulins which bind to the bio-agent are known.

In one embodiment, step d) comprises the comparison of the data obtained from the signal with data obtained from analysis of the total concentration of bio-agent present in the sample (or an equivalent sample). The concentration of bio-agent present in the sample may be predicted (e.g. by knowledge of the dosage and pharmacological properties of the bio-agent within the subject), or be measures—e.g. using an antibody based quantification assay.

### Suitable Bio-Agents and Disorders

An extensive list of monoclonal antibody therapeutics in clinical development and approved products are disclosed in the 2006 PhRMA Report entitled ‘418 Biotechnology Medicines in Testing Promise to Bolster the Arsenal Against Disease’. It is considered that the present invention may be used against these as well as other single-light chain monoclonal antibodies used as therapeutics or in vivo diagnostics. See table 1 for examples of monoclonal antibodies, which have either been approved or are currently in development.

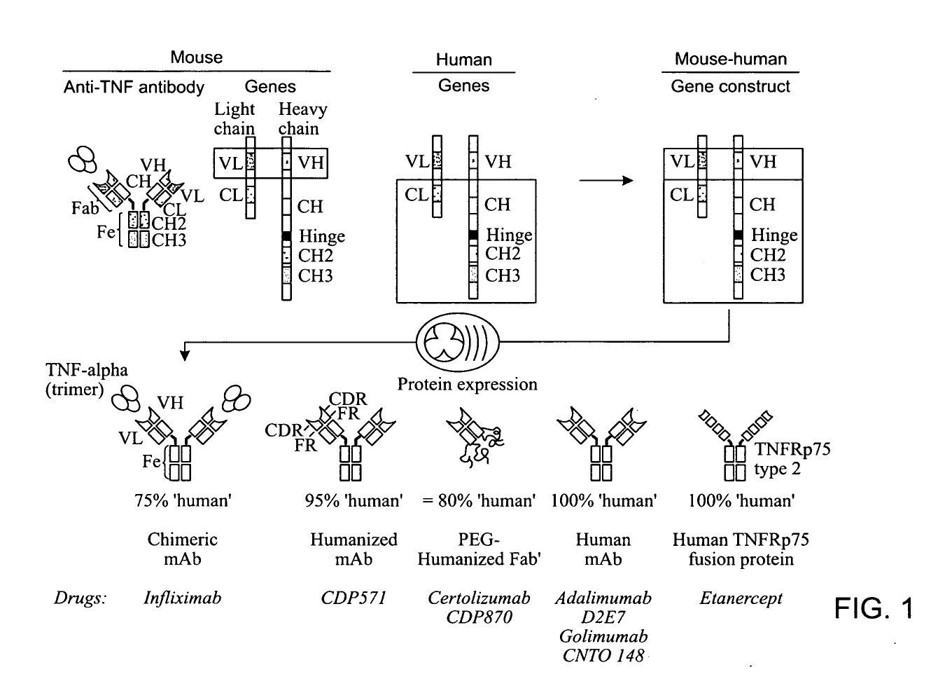

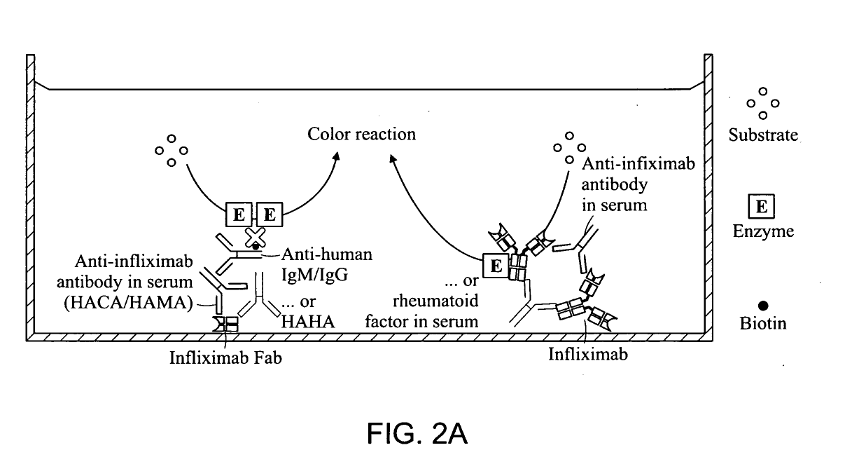

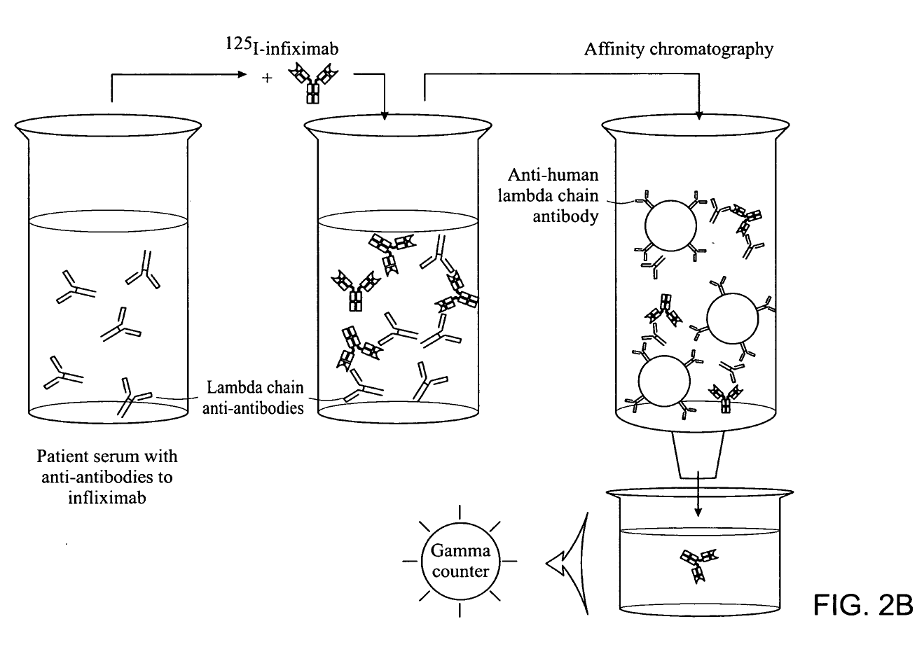

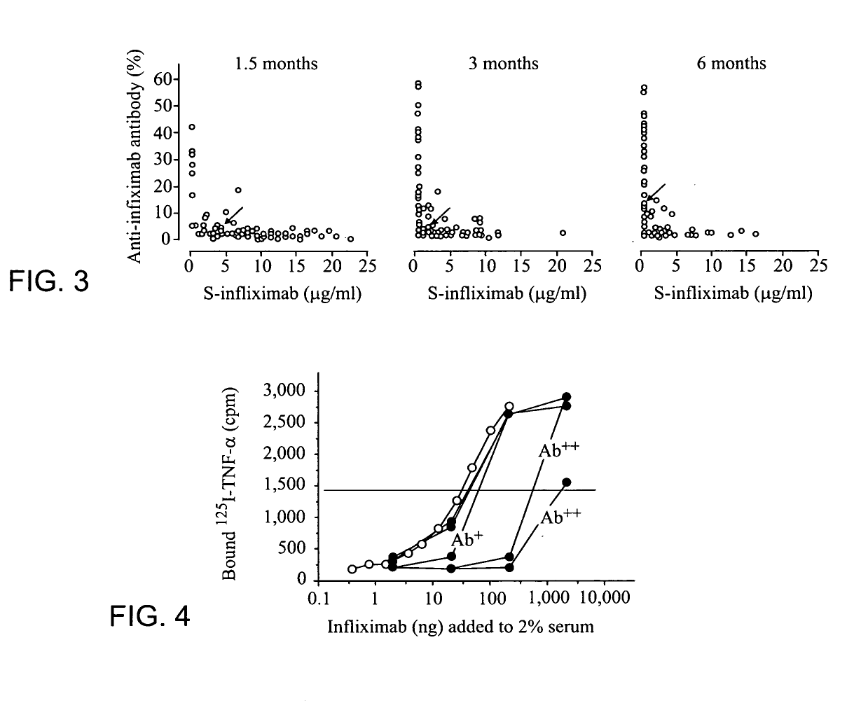

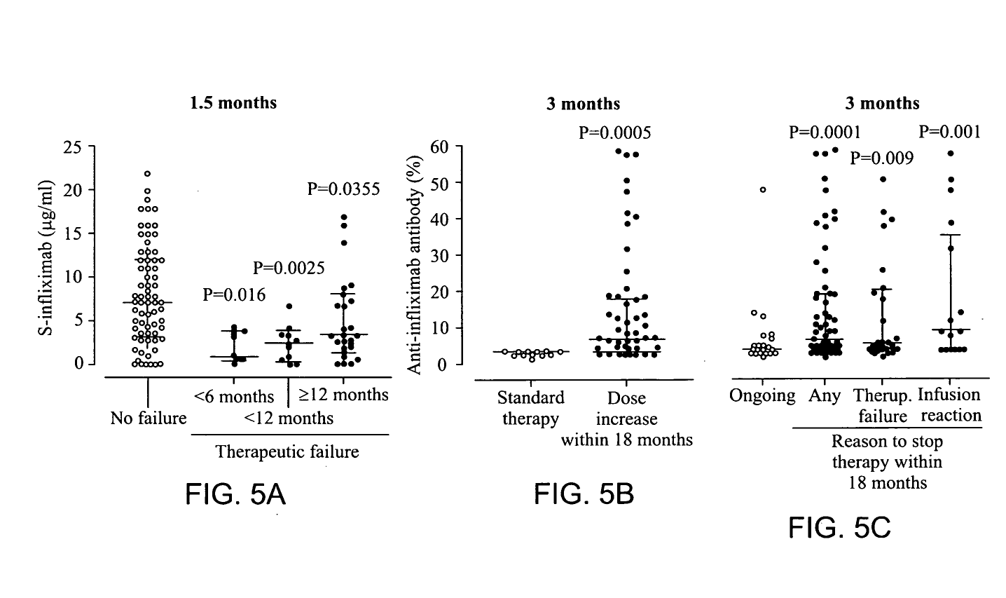

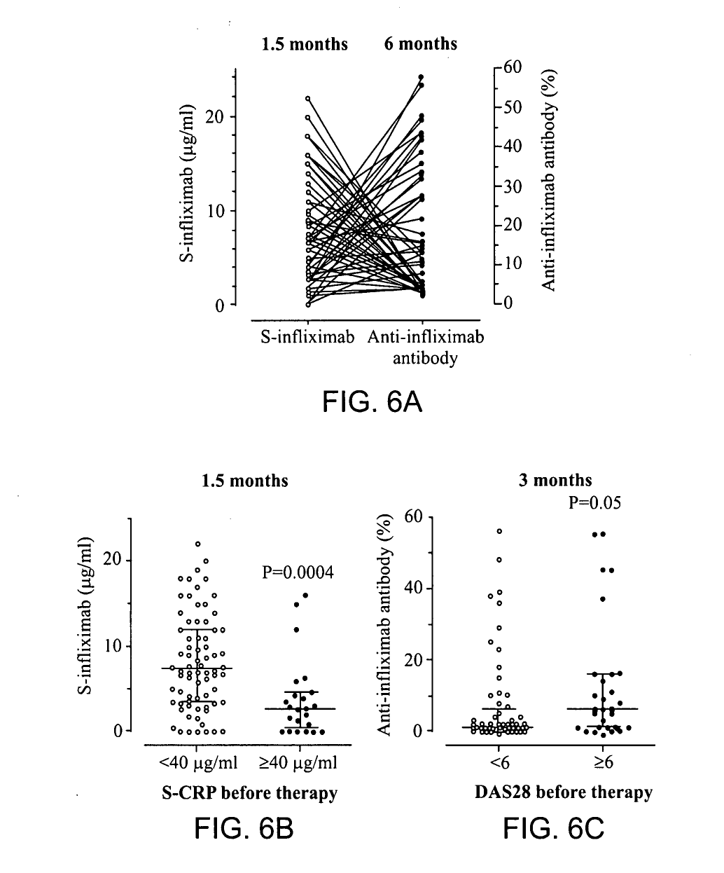

Particularly preferred bio-agents are the anti-TNFalpha monoclonal antibodies, which include (see FIG. 1) Remicade™ (infliximab), a mouse-human IgG1-kappa anti-TNF-alpha monoclonal antibody, 2) Enbrel™ (etanercept), a fusion protein of human TNF receptor 2 and human IgG1, and 3) Humira™ (adalimumab), a fully human IgG1-kappa anti-TNF-alpha monoclonal antibody. Two other anti-TNF-alpha antibody constructs have shown promise in pivotal phase III trials in patients with some of the same diseases: 4) Cimzia™ CDP870 (certolizumab pegol), a PEGylated Fab fragment of a humanized anti-TNF-alpha monoclonal antibody, and 5) CNTO 148 (golimumab), a fully human IgG1-kappa anti-TNF-alpha monoclonal antibody.

A preferred class of bio-agents are anti-TNF-alpha single chain monoclonal antibodies which are used in treatment of numerous autoimmune diseases, such as—rheumatoid arthritis, juvenile idiopathic arthritis, ankylosing spondylitis (Bechterew's disease), inflammatory bowel diseases (Crohn's diseases and ulcerative colitis), severe psoriasis, chronic uveitis, severe sarcoidosis and Wegener's granulomatosis.

Whilst is it recognised that the present invention is particularly useful in determining the bioavailability/concentration of anti-TNF-alpha alpha single chain monoclonal antibodies—it is clear that the present invention is suitable for use in the determination of the bioavailability/concentration of any single chain monoclonal antibody which is used within the body, such as for therapeutic or diagnostic purposes. Table 1 also provides a list of medical indications which correlated to various monoclonal bio-agents used in vivo.

The present invention can therefore be use in a method of treatment where the treatment (or diagnosis) comprises administering a single chain monoclonal antibody to the subject. Suitably the method can be for the treatment or diagnosis of one or more of the disorders/diseases referred to herein, including one or more of the following:

Infectious diseases, such as respiratory syncytial virus (RSV), HIV, anthrax, candidiasis, staphylococcal infections, hepatitis C

Autoimmune diseases, such as rheumatoid arthritis, Crohn's disease, B-cell non hodgkin's lymphoma, Multiple scleorisis, SLE, ankylosing spondylitis, lupus, psoriatic arthritis, erythematosus.

Inflammatory disorders such as rheumatoid arthritis (RA), juvenile idiopathic arthritis, ankylosing spondylitis (Bechterew's disease), inflammatory bowel diseases (Crohn's diseases and ulcerative colitis), severe psoriasis, chronic uveitis, sarcoidosis, Wegener's granulomatosis, and other diseases with inflammation as a central feature.

Blood disorders, such as sepsis, septic shock, paroxysmal nocturnal hemoglobinuria, and hemolytic uremic syndrome.

Cancer, such as colorectal cancer, non-Hodgkin's lymphoma, B-cell chronic lymphocytic leukemia, anaplastic large-cell-lymphoma, squamous cell cancer of the head and neck, treatment of HER2-overexpressing metastatic breast cancer, acute myeloid leukemia, prostate cancer (e.g. adenocarcinoma), small-cell lung cancer, thyroid cancer, malignant melanoma, solid tumors, breast cancer, early stage HER2-positive breast cancer, first-line non-squamous NSCLC cancers, AML, hairy cell leukemia, neuroblastoma, renal cancer, brain cancer, myeloma, multiple myeloma, bone metastases, SCLC, head/neck cancer, first-line pancreatic, SCLC, NSCLC, head and neck cancer, hematologic and solid tumors, advanced solid tumors, gastrointestinal cancer, pancreatic cancers, cutaneous T-cell lymphoma, non-cutaneous T-cell lymphoma, CLL, ovarian, prostate, renal cell cancers, mesothelin-expressing tumors, glioblastoma, metastatic pancreatic, hematologic malignancies, cutaneous anaplastic large-cell MAb lymphoma, AML, myelodysplastic syndromes.

Cardiovascular disease, such as atherosclerosis acute myocardial infarction, cardiopulmonary bypass, angina.

Metabilic disorders such as diabetes, such as type-1 diabetes mellitus

Digestive disorders, such as Crohn's disease, C. difficile disease, ulcerative colitis

Eye disorders such as uveitis.

Genetic Disorders such as paroxysmal nocturnal hemoglobinuria (PNH)

Neurological Disorders such as osteoarthritis pain and Alzheimer's disease.

Respiratory Disorders such as respiratory diseases, asthma, chronic obstructive pulmonary disorders (COPD, nasal polyposis, pediatric asthma.

Skin diseases, such as psoriasis, including chronic moderate to severe plaque psoriasis.

Transplant rejection, such as acute kidney transplant rejection, reversal of heart and liver transplant rejection, prevention of renal transplant rejection, prophylaxis of acute kidney transplant rejection, renal transplant rejection.

Other disorders, such as diagnosis of appendicitis, kidney inflammation postmenopausal osteoporosis (bone disorders), hypereosinophilic syndrome, eosinophilic esophagitis and peanut allergy.

In one embodiment the disease is selected from one or more of the above groups or specific diseases/disorder. Preferred diseases are diseases where repeated dosages of the bio-agent are used, such as autoimmune diseases.

TABLE 1
Therapeutic and diagnostic monoclonal antibodies (Approved are underlined)

| | | |
|---|---|---|
| Product | | |
| Name | Sponsor | Indication |

Infectious diseases

| | | |
|---|---|---|
| Synagis ® | MedImmune | prevention of respiratory syncytial virus |
| palivizumab | | (RSV) |
| anti-HIV-1 MAb | Polymun Scientific | HIV infection treatment |
| | Vienna, Austria | |
| CCR5 MAb | Human Genome | HIV infection |
| | Sciences | |
| | Rockville, MD | |
| Cytolin ® | CytoDyn | HIV infection |
| anti-CD8 MAb | Santa Fe, NM | |
| NM01 | SRD Pharmaceuticals | HIV infection |
| | Los Angeles, CA | |
| PRO 140 | Progenics | HIV infection |
| | Pharmaceuticals | |
| | Tarrytown, NY | |
| TNX | 355 Tanox MAb HIV | 355 Tanox MAb HIV infection Phase II |
| | infection Phase II | |
| ABthrax ™ | Human Genome | anthrax |
| raxibacumab | Sciences | |
| Anthim ™ | Elusys Therapeutics | anthrax |
| (ETI-204) | | |
| (Orphan Drug) | | |
| anti-hsp90 MAb | NeuTec Pharma | candidiasis |
| anti-staph MAb | MedImmune | Prevention of staphylococcal infections |
| Aurexis | Inhibitex | prevention and treatment of S. aureus |
| tefibazumab | | bacteremia |
| bavituximab | Peregrine | hepatitis C treatment |
| | Pharmaceuticals | |
| MDX-1303 | Medarex | anthrax |
| | PharmAthene | |
| Numax ™ | MedImmune | RSV |
| motavizumab | | |
| Tarvacin ™ | Peregrine | hepatitis C |
| bavituximab | Pharmaceuticals | |
| XTL 6865 | XTL Biopharmaceuticals | hepatitis C |

Autoimmune disorders

| | | |
|---|---|---|
| Humira ® | Abbott Laboratories | rheumatoid arthritis |
| adalimumab | | |
| Remicade ™ | Centocor | Crohn's disease, rheumatoid arthritis |
| infliximab | | |
| Rituxan ® | Genentech | B-cell non hodgkin's lymphoma, relapse |
| ritiximab | Biogen Idec | in patients following rituxan treatment. |
| | | Rheumatoid arthritis |
| Tysarbi ® | Biogen Idec | Multiple scleorisis |
| natalizumab | | |
| ABT 874 | Abbott Laboratories | multiple sclerosis, |
| Actemra | Roche | rheumatoid arthritis, |
| AME 527 | Applied Molecular | rheumatoid arthritis |
| AMG 108 | Amgen | rheumatoid arthritis |
| AMG 714 | Amgen | rheumatoid arthritis |
| anti-CD16 MAb | MacroGenics | immune thrombocytopenic |
| CNTO 1275 | Centocor | multiple sclerosis |
| | Horsham, PA | |
| daclizumab | PDL BioPharma | multiple sclerosis |
| (anti-CD25 MAb) | Fremont, CA | (see also respiratory) |
| | Biogen Idec | |
| | Cambridge, MA | |
| denosumab | Amgen | rheumatoid arthritis |
| (AMG 162) | Thousand Oaks, CA | |
| ETI-201 | Elusys Therapeutics | SLE |
| | Pine Brook, NJ | |
| golimumab | Centocor | rheumatoid arthritis |
| | Horsham, PA | |
| HuMax-CD20 | Genmab | rheumatoid arthritis |
| (ofatumumab) | Princeton, NJ | |
| Humira ® | Abbott Laboratories | ankylosing spondylitis |
| adalimumab | | juvenile rheumatoid arthritis |
| HuZAF ™ | PDL BioPharma | rheumatoid arthritis |
| fontolizumab | Fremont, CA | |
| | Biogen Idec | |
| | Cambridge, MA | |
| IMMU-106 | Immunomedics | autoimmune disease |
| (hCD20) | Morris Plains, NJ | |
| LymphoStat-B ™ | Human Genome | rheumatoid arthritis, SLE |
| belimumab | Sciences | |
| | Rockville, MD | |
| MEDI-545 | Medarex | lupus |
| (MDX-1103) | Princeton, NJ | |
| | MedImmune | |
| | Gaithersburg, MD | |
| MLN 1202 | Millennium | multiple sclerosis |
| | Pharmaceuticals | |
| | Cambridge, MA | |
| ocrelizumab | Genentech | rheumatoid arthritis |
| (2nd anti-CD20) | South San Francisco, | |
| (R1594) | CA | |
| | Biogen Idec | |
| | Cambridge, MA | |
| | Roche | |
| | Nutley, NJ | |
| OKT3-gamma-1 | Johnson & Johnson | psoriatic arthritis |
| | Pharmaceutical | |
| | Research | |
| | & Development | |
| | Raritan, NJ | |
| Rituxan ® | Genentech | rheumatoid arthritis |
| rituximab | South San Francisco, | (DMARD inadequate |
| | CA | responders), lupus, |
| | Biogen Idec | primary progressive |
| | Cambridge, MA | multiple sclerosis, SLE |
| | | (see also cancer) relapsing-remitting |
| | | multiple sclerosis |
| TRX 1 | TolerRx | cutaneous lupus |
| (anti-CD4) | Cambridge, MA | erythematosus |

Blood disorders

| | | |
|---|---|---|
| ReoPro ® | Centocor | anti-platelet prevention of blood clots |
| abciximab | Eli Lilly | (PTCA), angina (PTCA) |
| urtoxazumab | Teijin Pharma | hemolytic uremic |
| Afelimomab | Abbot Laboratories | Sepsis, septic shock |
| Eculizumab | Alexion | Paroxysmal nocturnal hemoglobinurea. |
| | Pharmaceuticals | |

Cancer

| | | |
|---|---|---|
| Avastin ™ | Genentech | metastatic colorectal cancer |
| bevacizumab | | |
| Bexxar ® | GlaxoSmithKline | non-Hodgkin's lymphoma |
| tositumomab, | | |
| iodine I 131 | | |
| tositumomab | | |
| Campath ® | Berlex Laboratories | B-cell chronic lymphocytic leukemia |
| alemtuzumab | Genzyme | |
| Erbitux ™ | Bristol-Myers Squibb | colorectal cancer |
| cetuximab | Medarex | squamous cell cancer of the head and |
| | | neck |
| Herceptin ® | Genentech | treatment of HER2-overexpressing |
| trastuzumab | | metastatic breast cancer |
| Mylotarg ™ | Wyeth | Acute myeloid leukemia |
| gemtuzumab | | |
| ozogamicin | | |
| OncoScint ® | CYTOGEN | detection, staging and follow-up of |
| CR/OV | | colorectal cancers |
| satumomab | | |
| pendetide | | |
| ProstaScint ® | CYTOGEN | detection, staging and follow-up of |
| capromab | | prostate adenocarcinoma |
| pentetate | | |
| Rituxan ® | Genentech | B-cell non hodgkin's lymphoma, relapse |
| ritiximab | Biogen Idec | in patients following rituxan treatment. |
| Verluma ® | DuPont | detection of small-cell lung cancer |
| nofetumomab | Pharmaceuticals | |
| Zevalin ™ | IDEC Pharmaceuticals | Non-hodgkin's lymphoma |
| ibritumomab | | |
| tiuxetan | | |
| 1311-huA33 | Life Science | colorectal cancer |
| | Pharmaceuticals | |
| | Greenwich, CT | |
| 1D09C3 | GPC Biotech | relapsed/refractory |
| | Waltham, MA | B-cell lymphomas |
| AGS-PSCA MAb | Agensys | prostate cancer |
| | Santa Monica, CA | |
| | Merck | |
| | Whitehouse Station, NJ | |
| AMG 102 | Amgen Thousand Oaks, | cancer |
| | CA | |
| AMG 479 | Amgen Thousand Oaks, | cancer |
| | CA | |
| AMG 623 | Amgen Thousand Oaks, | B-cell chronic lymphocytic leukemia |
| | CA | (CLL) (see also autoimmune) |
| AMG 655 | Amgen Thousand Oaks, | cancer |
| | CA | |
| AMG 706 | Amgen Thousand Oaks, | imatinib-resistant GIST, advanced |
| | CA | thyroid cancer |
| anti-CD23 MAb | Biogen Idec Cambridge, | CLL |
| | MA | |
| anti-CD80 MAb | Biogen Idec Cambridge, | non-Hodgkin's B-cell lymphoma |
| | MA | |
| anti-idiotype | Viventia Biotech | malignant melanoma |
| cancer vaccine | Toronto, Ontario | |
| anti-lymphotoxin | Biogen Idec Cambridge, | solid tumors |
| beta receptor | MA | |
| MAb | | |
| anti-PEM MAb | Somanta | cancer |
| | Pharmaceuticals Irvine, | |
| | CA | |
| anti-Tac(Fv)- | National Cancer | leukemia, lymphoma |
| PE38 | Institute Bethesda, MD | |
| immunotoxin | | |
| Avastin ® | Genentech | relapsed metastatic |
| bevacizumab | South San Francisco, | colorectal cancer |
| | CA | first-line metastatic breast, |
| | | first-line non-squamous NSCLC cancers |
| AVE 9633 | sanofi-aventis | AML |
| maytansin- | Bridgewater, NJ | |
| loaded | | |
| anti-CD33 MAb | | |
| bavituximab | Peregrine | solid cancers (see also infectious) |
| | Pharmaceuticals Tustin, | |
| | CA | |
| CAT 3888 | Cambridge Antibody | hairy cell leukemia |
| | Technology | |
| chimeric MAb | National Cancer | neuroblastoma |
| | Institute | |
| CNTO 328 | Centocor | renal cancer |
| Cotara ™ | Peregrine | brain cancer |
| | Pharmaceuticals | |
| bivatuzumab | Boehringer Ingelheim | cancer |
| | Pharmaceuticals | |
| | Ridgefield, CT | |
| CP-751,871 | Pfizer | multiple myeloma |
| CS 1008 | Daiichi Sankyo | cancer |
| | Sankyo Pharma | |
| | Development | |
| | Parsippany, NJ | |
| BrevaRex ™ | ViRexx | breast cancer, multiple |
| antibody-based | Edmonton, Alberta | myeloma |
| immunotherapy | | |
| denosumab | Amgen | bone loss induced by hormone ablation |
| | | therapy for breast or prostate |
| | | cancer, prolonging bonemetastases- |
| | | free survival |
| | | (see also autoimmune, other) |
| | | bone metastases in |
| | | breast cancer |
| ecromeximab | Kyowa Hakko USA | malignant melanoma |
| EMD 273063 | EMD Lexigen | solid tumors malignant melanoma, |
| | | neuroblastoma, SCLC |
| Erbitux ™ | Bristol-Myers Squibb | head/neck cancer, first-line pancreatic, |
| | | first-line NSCLC, -second-line NSCLC, |
| | | first |
| | | line colorectal, second-line |
| | | colorectal cancers |
| GMK | Progenics | prevention of recurrence following |
| | Pharmaceuticals | surgery to remove primary melanoma |
| | | in high-risk patients |
| Campath ® | National Cancer | leukemia, lymphoma |
| alemtuzumab | Institute | |
| | Bethesda, MD | |
| | Berlex Laboratories | |
| | Montville, NJ | |
| Herceptin ® | Genentech | early stage HER2-positive |
| trastuzumab | South San Francisco, | breast cancer |
| | CA | first-line metastatic HER2- |
| | | positive breast cancer in combination |
| | | with Taxotere ® |
| HGS-ETR1 | Human Genome | hematologic and |
| | Sciences | solid tumors |
| | Rockville, MD | |
| HGS-ETR2 | Human Genome | hematologic and |
| (mapatumumab) | Sciences | solid tumors |
| | Rockville, MD | |
| HGS-TR2J | Human Genome | advanced solid tumors |
| | Sciences | |
| | Rockville, MD | |
| HuC242-DM4 | ImmunoGen | colorectal, gastrointestinal, |
| | Cambridge, MA | NSCLC, pancreatic |
| | | cancers |
| HuMax-CD4 | Genmab | cutaneous T-cell |
| (zanolimumab) | Princeton, NJ Serono | lymphoma |
| | Rockland, MA | non-cutaneous T-cell |
| | | lymphoma |
| HuMax-CD20 | Genmab | CLL, non-Hodgkin's |
| (ofatumumab) | Princeton, NJ | lymphoma |
| | | (see also autoimmune) |
| HuMax-EGFr | Genmab | head and neck cancer |
| | Princeton, NJ | |
| huN901-DM1 | ImmunoGen | SCLC |
| | Cambridge, MA | multiple myeloma |
| ipilimumab | Bristol-Myers Squibb | melanoma monotherapy |
| (MDX-010) | Medarex, Princeton, | leukemia, lymphoma, ovarian, prostate, |
| | | renal cell cancers |
| | | melanoma |
| | | (MCX-010 +/− DTIC) |
| | | second-line metastatic |
| | | melanoma |
| | | (MDX-010 disomotide/ |
| | | overmotide MDX-1379) |
| M195-bismuth | Actinium | AML |
| 213 conjugate | Pharmaceuticals | |
| | Florham Park, NJ | |
| M200 | PDL BioPharma | advanced solid tumors |
| (volociximab) | Fremont, CA Biogen | |
| | Idec Cambridge, MA | |
| MAb HeFi-1 | National Cancer | lymphoma, non-Hodgkin's lymphoma |
| | Institute Bethesda, MD | |
| MDX-060 | Medarex Princeton, NJ | Hodgkin's disease, anaplastic large-cell- |
| (iratumumab) | | lymphoma |
| MDX-070 | Medarex Princeton, NJ | prostate cancer |
| MDX-214 | Medarex | EGFR-expressing cancers |
| | Princeton, NJ | |
| MEDI-507 | MedImmune | T-cell lymphoma |
| siplizumab | Gaithersburg, MD | infections |
| MEDI-522 | MedImmune | melanoma, prostate cancer |
| | Gaithersburg, MD | solid tumors |
| | National Cancer | |
| | Institute | |
| | Bethesda, MD | |
| | MedImmune | |
| | Gaithersburg, MD | |
| MORAb 003 | Morphotek | ovarian cancer |
| | Exton, PA | |
| MORAb 009 | Morphotek | mesothelin-expressing |
| | Exton, PA | tumors |
| neuradiab | Bradmer | glioblastoma |
| | Pharmaceuticals | |
| | Louisville, KY | |
| nimotuzumab | YM Biosciences | metastatic pancreatic, |
| (Orphan Drug) | Mississauga, Ontario | NSCLC |
| ocrelizumab | Genentech | hematologic malignancies |
| (2nd anti-CD20) | South San Francisco, | (see also autoimmune) |
| (R1594) | CA | |
| | Biogen Idec | |
| | Cambridge, MA | |
| | Roche | |
| | Nutley, NJ | |
| Omnitarg ™ | Genentech | ovarian cancer |
| pertuzumab | South San Francisco, | |
| | CA | |
| OvaRex ® | ViRexx MAb Edmonton, | ovarian cancer |
| oregovomab | Alberta | |
| PAM 4 | Merck Whitehouse | pancreatic cancer |
| | Station, NJ | |
| panitumumab | Abgenix | colorectal cancer |
| (rHuMAb-EGFr) | | |
| Proleukin ® | Chiron Emeryville, CA | Non-hodgkin's lymphoma |
| PSMA | Progenics | prostate cancer |
| | Pharmaceuticals | |
| | Tarrytown, NY | |
| R1550 | Roche Nutley, NJ | metastatic breast cancer |
| RadioTheraCIM | YM BioSciences | glioma |
| | Mississauga, Ontario | |
| RAV 12 | Raven Biotechnologies | cancer |
| | South San Francisco, | |
| | CA | |
| Rencarex ® | Wilex Munich, Germany | renal cancer |
| G250 | | |
| Rituxan ® | Genentech | indolent non-Hodgkin's |
| rituximab | South San Francisco, | lymphoma induction |
| | CA | therapy |
| | Biogen Idec | (see also autoimmune) |
| | Cambridge, MA | relapsed or refractory CLL |
| SGN-30 | Seattle Genetics | cutaneous anaplastic large- |
| (Orphan Drug) | Bothell, WA | cell MAb lymphoma, |
| | | systemic anaplastic large- |
| | | cell lymphoma, Hodgkin's |
| | | disease |
| SGN-33 | Seattle Genetics | AML, myelodysplastic |
| (lintuzumab) | Bothell, WA | syndromes |
| SGN-40 | Seattle Genetics | CLL |
| | Bothell, WA | multiple myeloma, non- |
| | | Hodgkin's lymphoma |
| sibrotuzumab | Life Science | colorectal, head and neck, |
| | Pharmaceuticals | lung cancers |
| | Greenwich, CT | |
| Tarvacin ™ | Peregrine | solid tumors |
| bavituximab | Pharmaceuticals | (see also infectious) |
| | Tustin, CA | |
| ticilimumab | Pfizer New York, NY | metastatic melanoma |
| | | prostate cancer |
| TNX-650 | Tanox Houston, TX | Hodgkin's disease |
| Zevalin ™ | National Cancer | leukemia, lymphoma non-Hodgkin's |
| ibritumomab | Institute Bethesda | lymphoma |
| tiuxetan | Biogen, | |

Cardiovascular disease

| | | |
|---|---|---|
| MLN 1202 | Millennium | atherosclerosis |
| | Pharmaceuticals | (see also autoimmune) |
| | Cambridge, MA | |
| pexelizumab | Alexion | acute myocardial |
| | Pharmaceuticals | infarction, cardiopulmonary |
| | Cheshire, CT | bypass |
| | Procter & Gamble | |
| | Pharmaceuticals | |
| | Mason, OH | |

Diabetes and Related Conditions

| | | |
|---|---|---|
| anti-CD3 MAb | MacroGenics Rockville, | type-1 diabetes mellitus |
| | MD | |
| OKT3-gamma-1 | Johnson & Johnson | type-1 diabetes mellitus |
| | Pharmaceutical | |
| | Research | |
| | & Development | |
| TRX 4 | TolerRx | type-1 diabetes mellitus |
| (anti-CD3) | Cambridge, MA | |

Digestive Disorders

| | | |
|---|---|---|
| Remicade ™ | Centocor | Crohn's disease, |
| infliximab | | |
| ABT 874 | Abbott Laboratories | Crohn's disease |
| | Abbott Park, IL | (see also autoimmune) |
| CNTO 1275 | Centocor | Crohn's disease Phase II |
| | Horsham, PA | (see also autoimmune, skin) (610) 651-6000 |
| Humira ® | Abbott Laboratories | Crohn's disease Phase III |
| adalimumab | Abbott Park, IL | (see also autoimmune, skin) (847) 936-1189 |
| MDX-066 | Medarex | C. difficile disease |
| (CDA-1) | Princeton, NJ | |
| MDX-1100 | Medarex | ulcerative colitis |
| | Princeton, NJ | |
| MLN-02 | Millennium | ulcerative colitis |
| | Pharmaceuticals | |
| | Cambridge, MA | |
| Nuvion ® | PDL BioPharma | I.V. steroid-refractory |
| visilizumab | Fremont, CA | ulcerative colitis |
| | | Crohn's disease |
| Tysarbi ® | Biogen Idec | Crohn's disease |
| natalizumab | Cambridge, MA | |

Eye Conditions

| | | |
|---|---|---|
| golimumab | Centocor Horsham, PA | uveitis (see also autoimmune) |

Genetic Disorders

| | | |
|---|---|---|
| Soliris ™ | Alexion Pharmaceuticals | paroxysmal nocturnal |
| eculizumab | Cheshire, CT | hemoglobinuria (PNH) |
| (Orphan | | |
| Drug) | | |

Neurological Disorders

| | | |
|---|---|---|
| RN624 | Rinat Neuroscience | osteoarthritis pain |
| | South San Francisco, CA | |
| RN1219 | Rinat Neuroscience | Alzheimer's disease |
| | South San Francisco, CA | |

Respiratory Disorders

| | | |
|---|---|---|
| ABN 912 | Novartis Pharmaceuticals | asthma, chronic |
| | East Hanover, NJ | obstructive pulmonary |
| | | disorders (COPD) |
| ABX-IL8 | Amgen | COPD |
| | Thousand Oaks, CA | |
| AMG 317 | Amgen | asthma |
| | Thousand Oaks, CA | |
| daclizumab | Protein Design Labs | asthma |
| (anti-CD25 | Fremont, CA Roche | (see also autoimmune) |
| MAb) | Nutley, NJ | |
| MEDI-528 | MedImmune | asthma |
| anti-IL-9 MAb | Gaithersburg, MD | |
| mepolizumab | GlaxoSmithKline | asthma and nasal polyposis |
| (anti-IL5 MAb) | Philadelphia, PA | (see also other) |
| | Rsch. Triangle Park, NC | |
| TNX-832 | Tanox | respiratory diseases |
| | Houston, TX | |
| Xolair ® | Genentech | pediatric asthma |
| omalizumab | South San Francisco, CA | (see also other) |
| | Novartis Pharmaceuticals | |

Skin Disorders

| | | |
|---|---|---|
| Raptiva ® | Genentech | chronic moderate to severe plaque |
| efalizumab | XOMA | psoriasis |
| CNTO 1275 | Centocor | psoriasis |
| | | see also autoimmune, |
| | | digestive) |
| Humira ® | Abbott Laboratories | psoriasis |
| adalimumab | | see also autoimmune, |
| | | digestive) |
| TRX 4 | TolerRx | psoriasis |
| | | (see also diabetes) |

Transplatation

| | | |
|---|---|---|
| ORTHOCLONE | Ortho Biotech | acute kidney transplant rejection, reversal |
| OKT ® 3 | | of heart and liver transplant |
| muromonab- | | rejection |
| CD3 | | |
| Simulect ® | Novartis | prevention of renal transplant rejection |
| basiliximab | Pharmaceuticals | |
| Zenapax ® | Roche | prophylaxis of acute kidney transplant |
| daclizumab | Protein Design Labs | rejection |
| OKT3-gamma-1 | Johnson & Johnson | renal transplant rejection |
| | | (see also autoimmune, diabetes) |

Other

| | | |
|---|---|---|
| NeutroSpec ™ | Palatin Technologies | diagnosis of appendicitis |
| technetium | | |
| 99m | | |
| Tc | | |
| fanolesomab | | |
| CR 0002 | CuraGen | kidney inflammation |
| denosumab | Amgen | Postmenopausal osteoporosis, see also |
| (AMG 162) | | autoimmune and cancer |
| mepolizumab | GlaxoSmithKline | hypereosinophilic |
| (anti-IL5 MAb) | | syndrome, eosinophilic |
| | | esophagitis (see also respiratory) |
| Xolair ® | Genentech | peanut allergy(see also respiratory) |
| omalizumab | Tanox | |

### EXAMPLES

We have developed two radioimmunoassays (RIA's), one for functional serum infliximab levels, in the form of TNF-alpha binding afforded by infliximab, and another for antibodies. We have used these assays to study individual RA patients for bioavailability, relation between trough levels of infliximab and antibody development, effect of disease activity and methotrexate therapy on these variables, and their ability to predict response failure and infusion-related side-effects.

### Materials and Methods

### Patients

Arthritis patients initiating biologic therapy were followed according to the protocol of the South Swedish Arthritis Treatment Group (SSATG)[15]. The follow up of biologic treatments in Sweden is part of a nationwide study imposed by the Swedish authorities including the Medical Products Agency. All patients are informed orally and in writing and give their consent to participate. To allow for at least 6 months follow-up, we randomly selected anti-TNF-alpha-naïve RA patients initiating infliximab therapy between March 1999 and December 2004 at the Department of Rheumatology, Lund, Sweden. Only patients from this centre were part of the study. All patients born in months 2, 3, 5, 6, 8, 10 or 11 were selected from the SSATG register. Twelve of the randomized 118 patients were excluded because of missing blood samples at 1.5, 3, and 6 month follow up time points (Table 2). Data on previous and concomitant DMARD therapy, infliximab and prednisolone dosage, CRP, and disease activity score (DAS28) were retrieved. Further information gathered from the database included drug stop and its cause (adverse event, primary failure, secondary failure). An adverse event was classified as the main stop reason regardless of previous response to treatment. The protocol only allows for one stop reason. Secondary failure was defined when a patient initially responding to therapy was withdrawn because of non-response despite dose adjustment. Primary treatment failure was defined as a patient not ever responding to therapy, and not experiencing an adverse event. This classification was made prospectively by the treating physician and prior to analyses of patient sera. To facilitate this the SSATG protocol includes feed-back where several clinical, laboratory, and drug variables are displayed graphically as well as information on fulfillment of established response criteria. Formal levels of responses are not required in the SSATG protocol nor in the national recommendations in Sweden. This is important since such recommendations might lead to reporting bias. Data on infusion reactions occurring within 24 hours of infusion were also collected. These were further classified as ‘type I’ (anaphylactic/hypotension, bronchospasm, wheezing and/or urticaria), or ‘other’ (unspecific rash, arthralgia, headache, fatigue, myalgia, influenza-like symptoms, etc.). To identify possible late infusion reactions and dose changes not recorded in the database, the medical records of patients still on therapy were further reviewed in December 2005.

All sera were tested in a blinded fashion at BioMonitor ApS, Copenhagen, Denmark.

TABLE 2
Characteristics of the 106 patients at inclusion in the study.

| | Mean ± SD | | Percent |
|---|---|---|---|
| Age (years) | 57 ± 13 | Female | 70 |
| Disease duration | 11 ± 8.7 | RF positive (ever) | 83 |
| (years) | | | |
| Previous DMARD | 3.1 ± 1.7 | Monotherapy | 20 |
| (no.) | | | |
| DAS28 | 5.4 ± 1.3 | Combination therapy | 80 |
| Swollen 28 joint count | 9.5 ± 6.2 | (one or more DMARD): | |
| (0-28) | | | |
| Tender 28 joint count | 8.6 ± 7.3 | methotrexate | 63 |
| (0-28) | | | |
| Patient global | 61.5 ± 23.9 | sulphasalazine | 17 |
| (VAS 0-100) | | | |
| ESR (mm/hour) | 35.4 ± 26.7 | azathioprine | 6.6 |
| CRP (mg/L) | 31.6 ± 35.6 | cyclosporine | 5.7 |
| | | hydroxychloroquine | 4.7 |
| | | other | 8.4 |
| | | Prednisolone | 76 |

### Serum-Infliximab (TNF-Alpha Binding to Serum IgG)

This was carried out using 1% patient sera added 5,000 cpm/100 μl of ¹²⁵I-TNF-alpha (Perkin-Elmer Life Science, Boston, Mass.). After incubation, free and IgG-bound tracer were separated by addition of a rabbit anti-human Fc-gamma antibody (Dako, Glostrup, Denmark) and centrifugation. The pellet activity was measured using a Wallac 1470 automatic gamma counter (Allerød, Denmark). Infliximab (Schering-Plough, Farum, Denmark) was used as reference and the TNF-alpha binding capacities are expressed as infliximab equivalents. The detection limit of the assay was 0.4 mg infliximab/ml of full-serum; the inter- and intraassay variations were below 20 and 10%, respectively.

### Anti-Infliximab Antibodies

We took advantage of the fact that infliximab is an IgG construct consisting only of kappa light-chains and, hence, that anti-human lambda light-chain antibody can be used to distinguish between free infliximab and infliximab in complex with any class of lambda-containing human immunoglobulin. The assay was carried out using 1% serum added 4,000 cpm/100 ml of ¹²⁵I-infliximab (spec. act. 1-2×10⁵ cpm/ng). After incubation, free and immunoglobulin-bound (any isotype) tracer were separated by affinity chromatography using small columns of matrix-bound anti-human immunoglobulin lambda-chain antibody (Dako). The data are given as % bound cpm/total cpm added; the backgrounds were 3%, and the inter- and intraassay variations were below 20 and 10%, respectively.

### Results

### Clinical Characterization

There were no significant differences regarding age, gender, disease duration, RF status, previous and concomitant DMARD treatments in the randomly selected patients compared to the total of 201 infliximab-treated biologic naïve patients at the Dept. of Rheumatology in Lund. The infliximab dosage was in all cases the recommended 3 mg per kg but had to be increased because of insufficient efficacy in 57 patients between 3 and 12 months. Of the 33 patients still on infliximab in December 2005, only 11 remained on the dose regimen established within the first 3 months, while increased doses and/or shortened intervals between infusions occurred in 19 patients after the first treatment year and in four patients between 3 and 12 months. A total of 23 infusion reactions occurred in 22 patients and was the stop reason in 15 patients (Table 3).

TABLE 3
Clinical events per follow up period during treatment.

| | Treatment periods | | | | |
|---|---|---|---|---|---|
| | 0-3 | 3-6 | 6-12 | >12 | |
| | months | months | months | months | Total |
| All events | | | | | |
| dose increase | | 28 | 29 | 20 | 77 |
| infusion reactions | 4 | 5 | 7 | 7 | 23 |
| Events leading to | | | | | |
| treatment withdrawal | | | | | |
| Adverse events | 5 | 7 | 17 | 13 | 42 |
| infusion reactions | 2 | 4 | 5 | 4 | 15 |
| Treatment failure | 2 | 5 | 5 | 13 | 25 |
| primary failure | 2 | 5 | 5 | 5 | 17 |
| secondary failure | 0 | 0 | 0 | 7 | 7 |
| Miscellaneous* | 0 | 0 | 1 | 3 | 4 |

*Two wish for pregnancy, 1 long pause because of living abroad, 1 patient decision.

Development of Anti-Infliximab Antibodies and Association with Trough Serum-Infliximab Levels

None of the sera tested positive for TNF-alpha binding before start of therapy, and while only 1 patient tested positive for anti-infliximab antibodies before treatment (over 3× background binding), 11/85 (13%) were positive before the third infusion of infliximab (1-5 months), whereas 28/93 (30%) and 33/75 (44%) were positive after 3 and 6 months, respectively (FIG. 3). Furthermore, the increased incidence and levels of antibodies over time were accompanied by decreased trough levels of infliximab. Indeed, functional bioavailability of the drug in sera collected immediately before infusions varied considerably between patients, particularly at 1-5 months where the number of antibody-positive sera was still low (FIG. 3, X-axes).

The known interference by infliximab in assays for anti-infliximab antibodies [7, 12] was investigated in separate competition experiments using 12 antibody-positive sera (Svenson et al: unpublished observations). While infliximab-levels as low as 2 mg/ml reduced the binding of ¹²⁵I-infliximab by median 40% (ranges 0-76%) in 12 patient sera, more than 200 mg/ml of the drug was required to completely mask the presence of antibodies in our RIA (ranges 80-100%).

Interference by RF was tested by cross-titrations with 5 known RF-positive sera without anti-infliximab antibodies; none of these influenced the RIA read-out of anti-infliximab antibodies. Neutralization of TNF-alpha binding afforded by anti-infliximab antibodies was further quantitated as shown in FIG. 4. Sera from 5 patients treated for 6 months with infliximab were added various amounts of the drug and tested for resulting ¹²⁵I-TNF-alpha binding capacities. The two antibody-positive sera required 20-80 times more infliximab to achieve the same TNF-alpha binding as a pool of sera from 30 untreated healthy blood donors. The data also show that serum TNF-alpha binding attributed by other factors than infliximab, e.g. soluble TNF receptors, are negligible in both the normal and patient sera.

Sera from 5 patients treated for 6 months with infliximab were assessed (closed circles): 2 had high antibody activity (Ab⁺⁺), 1 had low antibody activity (Ab⁺), and 2 had no detectable antibody activity. The sera were compared with a serum pool from 30 untreated healthy individuals (open circles). The amounts of added infliximab corresponded to the levels observed in patient sera (FIG. 1). Horizontal line shows 50% of maximum TNF-alpha binding afforded by added infliximab.

To further characterize the antibodies, we used RIA's to test 12 antibody-positive sera drawn after 3 months of infliximab therapy for cross-binding to two other anti-TNF-alpha immunoglobulin constructs, etanercept (Enbrel®) and adalimumab (Humira®). The binding of these drugs was less than 0.001 times that of infliximab.

### Relations to Clinical Outcome

### Low Infliximab Bioavailability Predicts Therapeutic Failure

Bioavailability of infliximab before the third infusion varied considerably from patient to patient, and a substantial number of patients with low serum levels of infliximab at this early time point (1·5 months) experienced response failure within the observation period (FIG. 5A).

High Antibody Level Predicts Dose Increase and/or Stop of Therapy

Detection of antibodies at an early time point (3 months) was associated with later dose increases necessitated by inadequate clinical responses (FIG. 5B). In fact, none of the patients who did well on standard therapy had detectable antibodies at 3 months.

Early formed antibodies were also associated with later discontinuation of therapy (FIG. 5C). This was the case both in general and when discontinuation was heralded by therapeutic failure or infusion reactions. The same tendency was seen when testing antibodies after 6 months of therapy, whereas too few were antibody-positive after 1.5 months for meaningful comparison.

### Initial Factors Influencing Antibody Development

Patients with low trough levels of infliximab after two infusions were most prone to develop antibodies at a later time, whereas patients with high levels were not (FIG. 6A). This tendency showed a highly significant linear correlation.

The influence of inflammation and disease activity at treatment start is shown in FIG. 6B, where patients with pronounced disease activity judged by pretreatment plasma-CRP levels in the upper quartile exhibited lower trough levels of functional infliximab compared with patients with lower disease activities. This tendency disappeared when testing after 3 months of therapy.

Patients with pronounced disease activity judged by pretreatment clinical activity (DAS28) above the upper quartile displayed higher levels of antibodies compared with patients with lower disease activities (FIG. 6C). A similar tendency was seen when testing for antibodies at 6 months, although this was not statistically significant.

The frequency of antibody-positive patients who also received methotrexate was not significantly different from those who did not. The numbers of antibody-positives at 6 months were 20 of 50 (40%) versus 13 of 26 (50%). However, patients on methotrexate had slightly lower levels of antibodies than those without: Medians (quartiles)=11% (4, 43) vs. 5% (3, 20), P=0.037. Treatments with other DMARD (Table 2) and prednisolone did not significantly affect the antibody levels.

### DISCUSSION

Anti-TNF biopharmaceuticals, including infliximab, are increasingly used to control disease activity in patients with RA and other chronic immunoinflammatory diseases [5-13]. The clinical use of these drugs has to a large extent been developed on the basis of pivotal clinical trials, where different dose regimens, efficacy, safety and tolerability have been monitored. In clinical practice, however, patients may differ considerably from the average patient involved in randomized clinical trials [14]. Another problem with prolonged use of biopharmaceuticals, including anti-TNF-alpha biologicals, is the induction of antibodies against the therapeutic proteins. Even though this is an area of increased interest, it is still unclear whether anti-anti-TNF antibodies measured by the most commonly used techniques (mostly solid-phase assays such as ELISA) are ‘functional’ in that they interfere with the bioactivities of anti-TNF-alpha biologicals and/or relate to clinical manifestations such as infusion reactions and reduction of long-term efficacy [12]. Monitoring bioavailability and neutralizing antibodies during infliximab therapy may therefore help optimize dose regimens to individual patients, diminish the risk of serious side-effects and prevent continued and probably futile use of infliximab in patients with neutralizing antibodies.

We therefore developed a functional RIA for infliximab bioavailability in the form of TNF-alpha binding to serum IgG (infliximab) and a soluble-phase RIA for anti-infliximab antibodies, and used them to monitor RA sera collected before start of therapy and immediately before readministration of the drug at time points 1.5, 3 and 6 months after start of therapy (trough levels). The antibody test was based on the fact that infliximab is an antibody constructed solely of kappa-light chains and that any radioactive complex extracted by anti-human lambda light-chain antibodies would be infliximab bound to antibody (irrespective of heavy-chain isotype). We deliberately refrained from using solid-phase assays because of the risk of getting false-positive results due to rheumatoid factors and heterophilic antibodies, which may interfere with sandwich ELISA's and nonspecific binding to immunoglobulin other than infliximab [16, 17].

Monitoring of RA patients in our study shows that there are pronounced interindividual differences in trough TNF-alpha binding capacities. This was seen even though all patients were initially treated with the same dose of infliximab, 3 mg/kg, suggesting that the generally recommended dose may not be optimal in all patients, as has been suggested before [18]. The reason for the marked individual differences is obscure. Variable contents of natural or disease-associated TNF-alpha binding factors in sera, for example soluble TNF receptors, is an unlikely explanation because there was no measurable binding of TNF-alpha in sera from healthy individuals (FIG. 2) or in any of the pre-treatment patient sera; endogenous binding of TNF-alpha was therefore negligible compared to that of infliximab. However, as infliximab is administered intravenously, with full initial blood bioavailability, a central factor might be differences in consumption of the drug. Thus, the total TNF-alpha ‘load’ in the body (not only serum levels), and therefore the in vivo binding of drug, may vary considerably in patients with different pre-treatment disease activities. This is supported by the finding that patients with high initial disease activity, judged both clinically (DAS28) and biochemically (plasma-CRP), were the ones with the lowest trough levels of bioactive infliximab. Similar findings have been reported recently [19]. Interestingly, these patients were also most prone to develop antibodies at later time points and, eventually, response failure.

The problem of antibodies to anti-TNF biopharmaceuticals has been addressed in several reports, often with no clear relation between the development of antibodies and response failure [5, 9, 10, 20]. In our study, however, the development of antibodies, heralded by low pre-infusion serum-infliximab levels, was clearly associated with an increased risk of infusion reactions and subsequent treatment failure. There are several possible explanations for this discrepancy, one is the frequent use of ELISA for antibody measurement because nonspecific (low affinity) immunoglobulins and anti-IgG rheumatoid factors may bind to adsorbed infliximab, thus generating false-positive results [16, 17]. We have tried to overcome this problem by using RIA's which are not influenced by artifacts induced by solid-phase adsorption of proteins, and therefore better reflect the in vivo situation, and not affected by RF-positive sera. Indeed, our data agree with a recent finding of RIA-detected antibodies to the F(ab′)₂ fragment of infliximab in 22 of 51 (43%) RA patients [20]; in this study, patients without anti-F(ab′)₂ antibodies were also more often classified as responders compared with those with antibodies (69% vs. 36%).

Another problem is the confounding presence of infliximab when testing for anti-infliximab antibodies [12]. This is particularly problematic if sera are collected shortly after administration of the drug and if tests are being used that are particularly sensitive to the presence of infliximab. We have tried to diminish this problem by solely evaluating sera drawn immediately before infusions of infliximab. Even so, the levels of anti-infliximab antibodies may have been underestimated as competition experiments showed an impact on the detection of antibody by as little as 2 mg/ml of infliximab. Importantly, however, the frequency of false-negative read-outs in the present study is likely to be negligible as more than 10 times the peak trough levels of the drug was required to mask the presence of antibodies in our RIA (Svenson et al: unpublished observations). Underrating of antibody-positives might also occur if anti-infliximab immune complexes consisted solely of kappa-light chain antibodies. Although this may occur, it is probably a rare event because size-chromatography of ten 6-months sera with low TNF-alpha binding and no detectable anti-infliximab antibodies did not reveal ‘hidden’ antibody complexes. At any rate, formation of infliximab-containing immune complexes would be expected to radically increase drug clearance in antibody-positive patients.

We did not find that antibodies invoked by infliximab therapy reacted significantly with the two other, currently approved anti-TNF-alpha immunoglobulin constructs, etanercept (Enbrel®) and adalimumab (Humira®). Although this does not rule out that cross-reacting antibodies may appear after prolonged infliximab therapy, it agrees with previous observations that both etanercept and adalimumab are effective in at least some patients who have failed on infliximab [21, 22].

The influence of concomitant therapies on antibody development was marginal in contrast to some previous reports; discussed in [12]. Thus, we did not find that prednisolone treatment or DMARD, apart from methotrexate, had any major impact on incidence or antibody levels obtained from up to 6 months' exposure to the drug. The effect of methotrexate, though not very pronounced in our study, is in accordance with other studies [1, 2, 6, 23, 24]. This is usually attributed to the immunosuppressive effect of methotrexate. In view of the lack of effect of prednisolone and the importance of disease activity for antibody development, we would suggest that methotrexate's disease-modifying effect may be important as well. This agrees with recent findings that methotrexate significantly reduces antibody responses to polysaccharides but not to polypeptide antigens [25], and Kapetanovic et al: unpublished observations.

In conclusion, our study illustrates that early monitoring of individual RA patients can optimize infliximab treatment. In particular, patients with low trough levels of infliximab may benefit from early instituted increased dosage or shorter intervals between drug administration, and early antibody development should question the efficacy, safety and economy of continued treatment. Although the methods developed in the present study are of value for monitoring of RA patients, it is recognized that the principle behind the protocol is of general application for the monitoring of bio-availability of monoclonal antibody bio-agents and/or concentration of host generated anti-bioagent antibodies in a subject.

### REFERENCES

1. Maini R, St Clair E W, Breedveld F, et al. Infliximab (chimeric anti-tumour necrosis factor alpha monoclonal antibody) versus placebo in rheumatoid arthritis patients receiving concomitant methotrexate: a randomised phase III trial. ATTRACT Study Group. Lancet 1999; 354: 1932-1939.
2. Lipsky P E, van der Heijde D M, St Clair E W, et al. Infliximab and methotrexate in the treatment of rheumatoid arthritis. N Eng J Med 2000; 343:1594-1602.
3. Keating G M, Perry C M. Infliximab: an updated review of its use in Crohn's disease and rheumatoid arthritis. BioDrugs 2002; 16:111-148.
4. Moreland L W. Drugs that block tumour necrosis factor: experience in patients with rheumatoid arthritis. Pharmacoeconomics 2004; 22:39-53.
5. Baert F, Noman M, Vermeire S, et al. Influence of immunogenicity on the long-term efficacy of infliximab in Crohn's disease. N Eng J Med 2003; 348:601-608.
6. Wagner C L, Schantz A, Barnathan E, et al. Consequences of immunogenicity to the therapeutic monoclonal antibodies ReoPro and Remicade. Dev Biol (BaseI) 2003; 112:37-53.
7. Sandborn W J. Optimizing anti-tumor necrosis factor strategies in inflammatory bowel disease. Curr Gastroenterol Rep 2003; 5:501-505.
8. Gottlieb A B, Masud S, Ramamurthi R, et al. Pharmacodynamic and pharmacokinetic response to anti-tumor necrosis factor-alpha monoclonal antibody (infliximab) treatment of moderate to severe psoriasis vulgaris. J Am Acad Dermatol 2003; 48:68-75.
9. Miele E, Markowitz J E, Mamula P, et al. Human antichimeric antibody in children and young adults with inflammatory bowel disease receiving infliximab. J Pediatr Gastroenterol Nutr 2004; 38:502-508.
10. Sands B E, Anderson F H, Bernstein C N, et al. Infliximab maintenance therapy for fistulizing Crohn's disease. N Eng J Med 2004; 350:876-885.
11. Feldmann M, Brennan F M, Foxwell B M, et al. Anti-TNF therapy: where have we got to in 2005? J Autoimmun 2005; 25 Suppl:26-28.
12. Cheifetz A, Mayer L. Monoclonal antibodies, immunogenicity, and associated infusion reactions. Mt Sinai J Med 2005; 72:250-256.
13. Anderson P, Louie J, Lau A, et al. Mechanisms of differential immunogenicity of tumor necrosis factor inhibitors. Curr Rheumatol Rep 2005; 7:3-9.
14. Flendrie M, Creemers M C, Welsing P M, et al. Survival during treatment with tumour necrosis factor blocking agents in rheumatoid arthritis. Ann Rheum Dis 2003; 62 Suppl 2: ii30-ii33.
15. Geborek P, Crnkic M, Petersson I F, et al. Etanercept, infliximab, and leflunomide in established rheumatoid arthritis: clinical experience using a structured follow up programme in southern Sweden. Ann Rheum Dis 2002; 61:793-798.
16. Hennig C, Rink L, Fagin U, et al. The influence of naturally occurring heterophilic anti-immunoglobulin antibodies on direct measurement of serum proteins using sandwich ELISAs. J Immunol Methods 2000; 235:71-80.
17. Bendtzen K. Anti-IFN BAb and NAb antibodies: A minireview. Neurology 2003; 61, Suppl. 5:56-S10.
18. St. Clair E W, Wagner C L, Fasanmade A A, et al. The relationship of serum infliximab concentrations to clinical improvement in rheumatoid arthritis. Results from ATTRACT, a multicenter, randomized, double-blind, placebo-controlled trial. Arthritis Rheum 2002; 46:1451-1459.
19. Wolbink G J, Voskuyl A E, Lems W F, et al. Relationship between serum trough infliximab levels, pretreatment C reactive protein levels, and clinical response to infliximab treatment in patients with rheumatoid arthritis. Ann Rheum Dis 2005; 64:704-707.
20. Wolbink G J, Vis M, Lems W, et al. Development of antiinfliximab antibodies and relationship to clinical response in patients with rheumatoid arthritis. Arthritis Rheum 2006; 54:711-715.
21. Haraoui B, Keystone E C, Thorne J C, et al. Clinical outcomes of patients with rheumatoid arthritis after switching from infliximab to etanercept. J Rheumatol 2004; 31:2356-2359.
22. Solau-Gervais E, Laxenaire N, Cortet B, et al. Lack of efficacy of a third tumour necrosis factor alpha antagonist after failure of a soluble receptor and a monoclonal antibody. Rheumatology (Oxford) 2006; 45:1121-1124.
23. Maini R N, Breedveld F C, Kalden J R, et al. Therapeutic efficacy of multiple intravenous infusions of anti-tumor necrosis factor a monoclonal antibody combined with low-dose weekly methotrexate in rheumatoid arthritis. Arthritis Rheum 1998; 41:1552-1563.
24. Gomez-Reino J J, Carmona L. Switching TNF antagonists in patients with chronic arthritis: an observational study of 488 patients over a four-year period. Arthritis Res Ther 2006; 8:R29.
25. Kapetanovic M C, Saxne T, Sjoholm A, et al. Influence of methotrexate, TNF blockers and prednisolone on antibody responses to pneumococcal polysaccharide vaccine in patients with rheumatoid arthritis. Rheumatology (Oxford) 2006; 45:106-111.

1. A method for determining the concentration or bioavailability of a single light chain subtype bio-agent in a biological sample obtained from a subject, said method comprising
a. Obtaining the single light chain subtype bio-agent in a form which comprises a detectable label;
b. admixing the biological sample with the labelled bio-agent and a probe which selectively binds the alternative single light chain subtype to form an incubation mixture;
c. isolating a fraction which is enriched for the probe and/or probe bound immunoglobulins from the incubation mixture;
d. measuring a signal from the detectable label present in the isolated fraction to determine the concentration or bioavailability of the single light chain subtype bio-agent in the biological sample.

2. The method according to claim 1, wherein the single light chain subtype bio-agent is a monoclonal antibody which comprises either the lambda or kappa single light chain sub-type, but not both lambda and kappa single light chain sub-types.

3. The method according to claim 1 or 2 where in the single light chain subtype bio-agent is either a humanised of a fully-human monoclonal antibody.

4. The method according to any one of claims 1-3 wherein the bio-agent is a biopharmaceutical.

5. The method according to claim 4 wherein the biopharmaceutical is an antibody which specifically binds TNF-alpha.

6. The method according to any one of claims 1-3 wherein the bio-agent is a bio-diagnostic.

7. The method according to any one of claims 1-6 wherein the biological sample is selected from the group consisting of blood, blood serum, lymph fluid, lymph node tissue, spleen tissue, bone marrow, or an immunoglobulin enriched fraction derived from one or more of these tissues.

8. The method according to any one of claims 1-7, wherein the probe which selectively binds the alternative single light chain subtype is an immunoglobulin, such as the alternative subtype to the sub-type referred to in claim 2.

9. The method according to any one of claims 1-8 wherein the detectable label is selected from the group consisting of: a radio label, a fluorescent label, and a luminescent label.

10. The method according to any one of claims 1-9, wherein the incubation step b) is performed in a fluid phase.

11. The method according to any one of claims 1-10, wherein step c) comprises a chromatographic step which enriches the probe and/or probe bound immunoglobulins on a basis of molecular size or affinity.

12. The method according to any one of claims 1-11 where step c) comprises an immuno-precipitation step

13. The method according to any one of claims 1-12 wherein the determination of the serum concentration or bioavailability in step b) is performed by comparing against control samples with predetermined or known concentration of the biopharmaceutical.

14. The method according to any one of claims 1-13 wherein said method comprises a radio-immuno assay (RIA).

15. A kit comprising:
a. A single light chain subtype biopharmaceutical in a form which comprises a detectable label,
b. A probe which selectively binds the alternative single light chain subtype to the biopharmaceutical.

16. A method of treatment of a disease with a single light chain subtype biopharmaceutical, said method comprising performing the method according to any one of claims 1-14 on a patient to determine whether the patient requires either an altered dosage regime of the biopharmaceutical or alternative pharmaceutical therapy.

17. The method according to claim 16, wherein the method comprises periodic assessment of the serum concentration or bioavailability of the biopharmaceutical in the patient.

18. The method according to claims 16 or 17, wherein the disease is selected from the group consisting of: rheumatoid arthritis (RA), juvenile idiopathic arthritis, ankylosing spondylitis (Bechterew's disease), inflammatory bowel diseases (Crohn's diseases and ulcerative colitis), severe psoriasis, chronic uveitis, sarcoidosis, Wegener's granulomatosis, and other diseases with inflammation as a central feature.

19. A method for determining the suitability or accuracy of an in vivo diagnostic method which involves the administration of a labelled bio-agent to a subject, said method comprising the method for determining the concentration or bioavailability of a single light chain subtype bio-agent in a biological sample obtained from a subject according to any one of claims 1-14, in order to determine either whether the patient a immune response to the bio-agent or the severity of the immune response.

20. A method for the measurement of host derived antibodies which recognise a single light chain subtype bio-agent in a biological sample obtained from a subject, said method comprising the following steps
a. Obtaining the single light chain subtype bio-agent in a form which comprises a detectable label;
b. admixing the biological sample with the labelled bio-agent and a probe which selectively binds the alternative single light chain subtype to form an incubation mixture;
c. isolating a fraction which is enriched for the probe and/or probe bound immunoglobulins from the incubation mixture formed in step b;
d. measuring a signal from the detectable label present in the isolated fraction to measure the level of host derived antibodies which recognise a single light chain subtype bio-agent in the biological sample.
